(19) United States
(12) Patent Application Publication
(10) Pub. No.: US 2025/0172529 A1
(43) Pub. Date: May 29, 2025

(54) ODOR DETECTION DEVICE, ODOR DETECTION METHOD, AND ODOR DETECTION PROGRAM

(71) Applicant: KONICA MINOLTA, INC., Tokyo (JP)

(72) Inventor: Masato SHINOMURA, Tokyo (JP)

(73) Assignee: KONICA MINOLTA, INC., Tokyo (JP)

(21) Appl. No.: 18/915,731

(22) Filed: Oct. 15, 2024

(30) Foreign Application Priority Data
Nov. 28, 2023 (JP) 2023-200425

(51) Int. Cl. G01N 33/00

(52) U.S. Cl. CPC G01N 33/0027

(57) ABSTRACT

The odor detection system of the present invention includes an odor sensor which reacts to the odor of an object, a speed calculation section which calculates the movement speed of the object as viewed from the odor sensor, and a determination part which determines the state of the object on the basis of the outputted value of the odor sensor and the movement speed of the object. Specifically, the odor determination system includes a plurality of odor determination models that have learned, for each of the plurality of values of movement speed, the output value of the odor sensor as an explanatory variable and the state of the object as an objective variable, and the determination section selects one of the odor determination models based on the movement speed calculated by the speed calculation section, and determines the state of the object using the selected odor determination model.

### CROSS REFERENCE TO THE RELATED APPLICATION

The entire disclosure of Japanese patent Application No. 2023-200425, filed on Nov. 28, 2023, is incorporated herein by reference in its entirety.

### BACKGROUND

### 1. Technological Field

The present invention relates to a technique for determining a state of an object using an odor.

### 2. Description of the Related Art

As a technique for detecting an odor, International Application Publication No. 2019/102660 discloses an odor detection device that specifies an odor component contained in a gas to be measured and the concentration of the odor component on the basis of output values of a plurality of odor sensors having different characteristics of reacting to odors.

### SUMMARY

The odor detection device described in International Application Publication No. 2019/102660 detects odor contained in gas around an odor sensor. When the odor of a moving object is to be measured, the determination result changes depending on the speed of the object to be measured passing near the sensor. That is, in a case where such an odor detection device is used for determining a state of an object that is a source of an odor, there is a problem that it is not possible to always correctly determine the state of the object when the movement speed of objects is different.

The present invention has been made in view of the above circumstances, and it is an object of the present invention to provide an odor detection system, an odor detection method, and an odor detection program capable of suitably determining a state of an object using an odor.

The present invention for solving the above-described problem includes the following configurations.

(1) An odor detection system including: an odor sensor that reacts to an odor of an object; a speed calculation section that calculates movement speed of the object as viewed from the odor sensor; and a determination section that determines a state of the object based on an output value of the odor sensor and the movement speed of the object.

(2) The odor detection system according to (1), wherein the determination section determines whether or not the object is abnormal.

(3) The odor detection system according to (2), wherein the determination section determines whether or not the object is damaged.

(4) The odor detection system according to (1), further including a plurality of odor determination models that have learned, for each of the plurality of values of movement speed, the output value of the odor sensor as an explanatory variable and the state of the object as an objective variable, wherein the determination section selects one of the odor determination models based on the movement speed calculated by the speed calculation section, and determines the state of the object using the selected odor determination model.

(5) The odor detection system according to (1), further including an odor determination model that has learned the movement speed and the output value of the odor sensor as explanatory variables and the state of the object as an objective variable, wherein the determination unit determines the state of the object from the movement speed calculated by the speed calculation section and the output value of the odor sensor using the odor determination model.

(6) The odor detection system according to (1), further including a distance calculation section that calculates a distance between the object and the odor sensor, wherein the determination section determines the state of the object on the basis of the output value of the odor sensor, the movement speed of the object, and the distance between the object and the odor sensor.

(7) The odor detection system according to (6), further including: a plurality of odor determination models that have learned, for each of a plurality of sets of the movement speed and the distance, an output value of the odor sensor as an explanatory variable and a state of the object as an objective variable, wherein the determination section selects one of the odor determination models based on the movement speed and the distance calculated by the speed calculation section, and determines the state of the object using the selected odor determination model.

(8) The odor detection system according to (6), further including an odor determination model that has learned the movement speed, the distance, and the output value of the odor sensor as explanatory variables and the state of the object as an objective variable, wherein the determination unit determines the state of the object based on the movement speed calculated by the speed calculation section, the distance calculated by the distance calculation section, and the output value of the odor sensor using the odor determination model.

(9) The odor detection system according to (1), further including a plurality of the odor sensors differing from each other in their characteristic of reacting to odor, wherein the determination section identifies the object based on output values of the plurality of odor sensors.

(10) An odor determination method, performed by a system including an odor sensor, a speed sensor, a speed calculation section and a determination section, the method allowing the system to perform: causing the odor sensor to respond to an odor of an object; causing the speed sensor to detect movement speed of the object; causing the speed calculation section to calculate movement speed of the object as viewed from the odor sensor; and causing the determination section to determine a state of the object on the basis of an output value of the odor sensor and the movement speed of the object as viewed from the odor sensor.

(11) A non-transitory computer-readable storage medium storing a program causing a computer to perform: acquiring an output value of an odor sensor that has reacted to an odor of an object; acquiring movement speed of the object detected by a speed sensor; calculating, by a speed calculation section, movement speed of the object as viewed from the odor sensor; and determining, by a determination section, a state of the object on the basis of the output value of the odor sensor and the movement speed of the object as viewed from the odor sensor.

### BRIEF DESCRIPTION OF THE DRAWINGS

Hereinafter, one or more embodiments of the present invention will be described with reference to the drawings. However, the scope of the invention is not limited to the disclosed embodiments.

FIG. 1 is a diagram of a configuration of an odor detection system according to a first embodiment.

FIG. 2A is a diagram schematically showing a method of generating a first odor determination model according to the first embodiment.

FIG. 2B is a diagram schematically showing a method of generating a second odor determination model according to the first embodiment.

FIG. 2C is a diagram schematically showing a method of generating a third odor determination model according to the first embodiment.

FIG. 3 is another configuration diagram of the odor detection system according to the first embodiment.

FIG. 4 is a flowchart for explaining an operation example of the odor detection system according to the first embodiment.

FIG. 5 is a diagram of the configuration of an odor detection system according to a second embodiment.

FIG. 6A is a diagram schematically showing a method of generating an odor determination model according to the second embodiment.

FIG. 6B is a table for explaining a method of generating an odor determination model according to the second embodiment.

FIG. 7 is a flowchart for explaining an operation example of the odor detection system according to the second embodiment.

FIG. 8A is a flowchart for explaining an operation example of the odor detection system according to the third embodiment.

FIG. 8B is a diagram illustrating a relationship among movement speed information, the degree of abnormality at that time, and optimal thresholds in an abnormality detection model.

FIG. 9 is a diagram of the configuration of an odor detection system according to a fourth embodiment.

FIG. 10 is a table for explaining a method of generating an odor determination model according to the fourth embodiment.

FIG. 11 is a flowchart illustrating an operation example of the odor detection system of the fourth embodiment.

FIG. 12 is a diagram of the configuration of an odor detection system according to a fifth embodiment.

FIG. 13 is a table for explaining a method of generating an odor determination model according to the fifth embodiment.

FIG. 14 is a flowchart illustrating an operation example of the odor detection system of the fifth embodiment.

FIG. 15 is a diagram for explaining a specific application example of the odor detection system of the embodiment.

### DETAILED DESCRIPTION OF THE EMBODIMENTS

Although embodiments of the present invention have been described and illustrated in detail, the disclosed embodiments are made for purposes of illustration and example only and not limitation. The scope of the present invention should be interpreted by terms of the appended claims.

Hereinafter, embodiments of the present invention will be described in detail with reference to the drawings. The same reference numerals will be given to the same constituent elements and the description of the redundant description will be omitted.

### First Embodiment

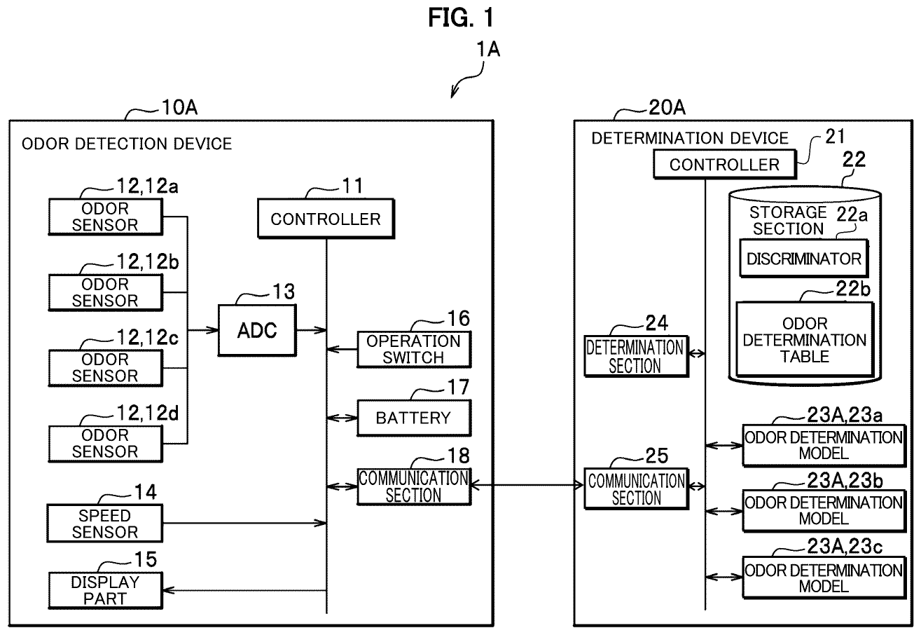

FIG. 1 is a configuration diagram of an odor detection system 2A according to a first embodiment, which determines a state of an object 2 (see FIG. 1A) based on an odor of the object and a speed at which the object moves.

Examples of the object 2 include food and drink (such as fruits and vegetables), human body odor, and the like. The state may be a level (for example, two stages of normal or abnormal) or a continuous value. Furthermore, examples of the abnormality include breakage of a container or packaging of food and drink (smell of food and drink is emitted), and rotten vegetables and fruits (emitting a rotten odor).

The odor detection system 1A includes an odor detection device 10A and a determination device 20A. The odor detection device 10A and the determination device 20A include a short-range wireless communication section 18 and 25, respectively, and can perform intercommunication by Bluetooth (R). The communication methods of the odor detection device 10A and the determination device 20A are not limited to those described above and may be wireless communication other than Bluetooth (R) or wired network communication such as LANs.

The odor detection device 10A includes a controller 11, a plurality of odor sensors 12 (12a, 12b, 12c, and 12d), an ADC (Analog to Digital Converter) 13, a speed sensor 14, a display part 15, an operation switch 16, a battery 17, and a communication unit 18.

The controller 11 is configured by CPUs (Central Processing Units) and the like, and integrally controls the processing operation of each unit of the odor detection device 10A by executing an odor detection program (not shown). Specifically, the controller 11 reads various processing programs stored in a storage section (not illustrated) and performs various kinds of processing in cooperation with the programs.

The odor sensors 12a to 12d are semi-conductor gas sensors different from each other in their characteristics of reacting to the odor, such as which odor the sensors strongly react to. The odor sensors 12a to 12d convert the concentration of a detection target gas (mainly a gas to be reacted) into an electrical quantity and output an electrical signal corresponding to the gas concentration. The odor sensors 12a to 12d may include, for example, a gas sensor for detecting volatile organic compounds (VOCs), a gas sensor for detecting volatile organic compounds), a gas sensor for detecting CO, a gas sensor for detecting hydrogen, a gas sensor for detecting hydrocarbons, a gas sensor for detecting alcohol, a gas sensor for detecting tobacco, and the like. Each of the odor sensors 12a to 12d responds to a plurality of odor components and does not respond to only one of the odor components. Here, the odor component is a chemical substance that forms an odor. Note that the odor sensors 12a to 12d may be micro electromechanical systems (MEMS) type sensors. In the present specification, a case where the odor sensor 12 is constituted by four sensors will be described, but it is needless to say that the odor sensor 12 is not limited thereto.

The ADC13 converts the analog signals outputted from the odor sensors 12a to 12d into digital signals, and outputs the converted digital signals to the controller 11.

The speed sensor 14 is a sensor that detects the speed of movement of the target object 2 for state determination, that is, a parameter related to the speed of movement of the target object 2 as viewed from the speed sensor 14. As the speed sensor 14, for example, a sensor using a laser, infrared rays, electromagnetic waves, ultrasonic waves, or the like can be used. Furthermore, in a case where the object 2 moves by a conveyance belt or the like, a moving object speed acquisition section that acquires a conveyance speed of the conveyance belt as the movement speed of the object 2 may be provided in place of the speed sensor 14.

The controller 11 functions as a speed calculation section that acquires a detection result (a parameter related to the speed of the target object 2) of the speed sensor 14 and calculates the speed of the target object 2 as viewed from the odor sensors 12a to 12d based on the acquired detection result.

The display part 15 is configured by a monitor or the like and displays the determination result of the state of the target object 2 of the determination device 20A, which will be described later, based on the control of the controller 11.

The operation switch 16 includes a power switch for turning on/off the power supply, a measurement switch for instructing the start of measurement, and the like. The operation switch 16 outputs an operation signal corresponding to the operation (pressing) of the operation switch 16 by the user to the controller 11.

The battery 17 supplies power to each part of the odor detection device 10A. As the battery 17, a detachable dry cell, a rechargeable battery, or the like is used.

The communication unit 18 includes an interface for performing wireless communication such as Bluetooth (R) with the terminal 40 and the determination apparatus 20A, which will be described later, and performs data transmission and reception with the determination apparatus 20A in accordance with a communication standard.

The determination device 20A is a server, a personal computer, or the like, and includes a controller 21, a storage section 22, a plurality of odor determination models 23A (23a, 23b, 23c), a determination unit 24, and a communication unit 25.

The controller 21 is configured of a CPU and the like. The controller 21 comprehensively controls the processing operation of each part of the determination device 20A by executing a determination program (not illustrated). Specifically, the controller 21 reads various processing programs stored in the storage section 22 and performs various processes in cooperation with the programs.

The storage section 22 is configured by a non-volatile semiconductor memory or the like, and stores various processing programs, parameters necessary for executing the programs, files, and the like.

The storage section 22 stores a discriminator 22a and an odor type determination table 22b.

The discriminator 22a is a machine learning result generated in advance by an external device in the development stage of the odor detection device 10A. At the time of machine learning, each of a plurality of odor components (chemical substances) is prepared for each concentration. The external device acquires, for each concentration of each of the plurality of odor components, values (waveforms) outputted by odor sensors of the same type as the odor sensors 12a to 12d (hereinafter referred to as odor sensors for machine learning). Then, the machine learning is performed to generate a discriminator 22a with using a combination of output values of the respective odor sensors for machine learning when the odor component of the concentration is set as a target as an input and the odor component and the concentration thereof as an output. That is, machine learning is performed using the set of data as teacher data, the set of data including a combination of output values of a plurality of odor sensors for learning as an input, and an odor component and a concentration thereof as an output.

“Odor sensors of the same type” refer to sensors having the same characteristics as the odor sensors 12a to 12d (sensors that can obtain the same output), and for example, are sensors of the same model number. As the machine learning model, for example, a neural network, particularly a learning vector quantization (LVQ) neural network is used. For example, the discriminator 22a is constructed by using, as the feature amount, a rising manner, a peak value, or the like of a waveform obtained by plotting the gas concentration, which is the output value of the odor sensor for machine learning, along the elapsed time from the start of measurement.

Examples of the odorous components include components that cause a malodor, such as “nonenal, diacetyl, isovaleric acid, and ammonia”. Nonenal is a component that causes aging odor (body odor that develops with aging). Diacetyl is a component that causes a middle fat odor (a greasy body odor common to middle-aged men). Isovaleric acid is a component that causes sweat odor (body odor due to sweat).

Note that here, the odor sensors for machine learning are used when the discriminator 22a is generated in advance, but machine learning may be performed using the odor sensors 12a to 12d themselves actually mounted in the odor detection device 10.

The odor type determination table 22b is a table in which, for each of a plurality of odor components, the type of odor and the intensity (level) thereof are associated with each other for each concentration. The odor type determination table 22b is information serving as a determination criterion for converting the concentration of each odor component into the type of odor (and its intensity). The types of odors are classified and named by a human being with respect to odors having some characteristics. Examples of the type of odor include the odor of food and drink as the target object 2, and three major odors of “aging odor, middle fat odor, and sweat odor”.

The storage section 22 also stores in advance, for each concentration of each of the odor components, values (waveforms) outputted from the plurality of odor sensors 12a to 12d when the odor components at the concentration are targets. Furthermore, in the storage section 22, values (waveforms) outputted from the plurality of odor sensors 12a to 12d when a predetermined odor is targeted are stored in advance. Data of an output value corresponding to each concentration of each odor component, stored in the storage section 22 and the data of the output value corresponding to the predetermined odor are obtained in advance using the odor sensor for learning. Note that the odor information may be obtained in advance by use of the odor sensors 12a to 12d themselves that are actually mounted in the odor detecting device 10.

Further, the name of the object 2 and the type of odor are stored in the odor type discrimination table 22b in association with each other.

The odor determination model 23a (23a to 23c) is a model that has learned, for each of a plurality of speed ranges, values (or feature amounts) outputted from the odor sensor 12 as explanatory variables and the state of the object 2 as an objective variable. The odor determination model 23A includes thresholds of the outputs of the odor sensor 12 obtained by machine learning. If the values outputted by the odor sensors 12a to 12c are less than the thresholds, it is determined that the object 2 is normal. If the outputs of the odor sensors 12a to 12c are equal to or greater than the thresholds, it is determined that the object 2 is abnormal. Hereinafter, three odor determination models will be described, but it is needless to say that the present invention is not limited thereto.

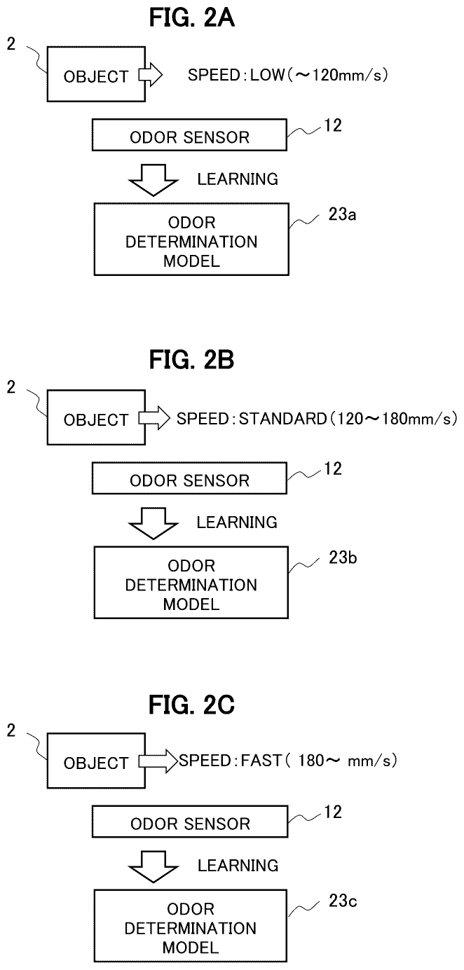

As illustrated in FIG. 2A, the odor determination model 23a is a model obtained by machine learning based on the outputs (or features) of the odor sensor 12 for the target object 2 that is located at a predetermined position from the odor sensor 12 and moves in a low speed range (0 to 120 mm/s).

As illustrated in FIG. 2B, the odor determination model 23b is a model obtained by machine learning based on the outputs (or feature amounts) of the odor sensor 12 for the target object 2 that is located at a predetermined position from the odor sensor 12 and moves in a standard range (120˜180 mm/s).

As illustrated in FIG. 2C, the odor determination model 23c is a model obtained by machine learning based on the outputs (or feature amounts) of the odor sensor 12 for the target object 2 which is located at a predetermined position from the odor sensor 12 and moves in a high speed range (180 to mm/s).

In the odor determination models 23a to 23c, the thresholds are individually set. When the speed of the object 2 is low, the odor reaches the odor sensor 12 relatively unattenuated, and therefore, the threshold value is set high to perform robust determination that has a low false alarm rate (a rate of determining normality as abnormality) and is less likely to be influenced by disturbance. When the speed of the object 2 is high, the odor is diluted before reaching the odor sensor 12, and thus the threshold value is set low to decrease the notification failure rate (the rate at which an abnormality is determined to be normal).

Referring back to FIG. 1, the determination section 24 is composed of a CPU and the like. The determination section 24 acquires the values outputted from the odor sensors 12a to 12d via the communication sections 18 and 25 and generates the feature amount of the odor on the basis of the acquired values outputted from the odor sensors 12a to 12d. Examples of the feature amounts include how a waveform rises which is obtained by plotting the gas concentrations that are the output values of the odor sensors 12a to 12d along the elapsed time from the start of the measurement and the peak value of the waveform.

Furthermore, the determination section 24 determines the odor component, and its concentration based on the outputs (or the feature amounts) from the odor sensors 12a to 12d and the identifier 22a.

In addition, the determination section 24 refers to the odor type determination table 22b using the calculated odor components and the concentrations thereof. Thus, the determination section 24 determines the odor type, and identifies the object 2 based on the determination result (by reading the name of the object 2 associated with the determined odor type).

The communicator 25 includes an interface for performing wireless communication such as Bluetooth (R) with the odor detection device 10A and the below-described terminals 40 and performs data transmission and reception with the odor detection device 10A in accordance with the communication standard.

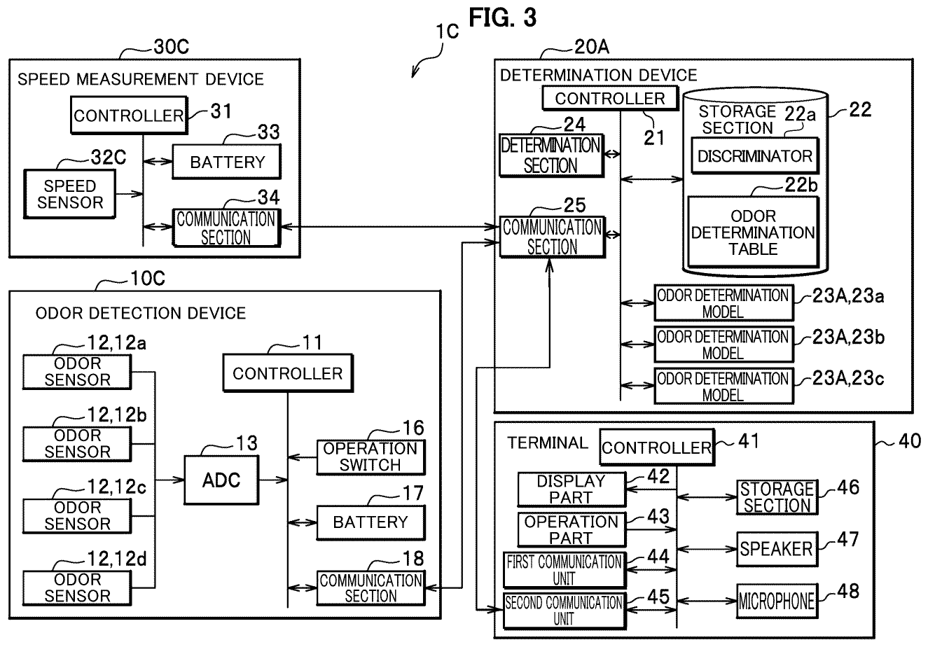

FIG. 3 is a diagram of another configuration of the odor detection system according to the first embodiment. The odor detection system of FIG. 3 is different from the odor detection system of FIG. 1 in that the speed measurement device 10A detects the speed of the target object 2 instead of the speed sensor 14 in the odor detection device 30C of the odor detection system of FIG. 1 and the determination result of the target object 2 is notified to the terminals 40.

The speed measuring device 30C includes a controller 31, a speed sensor 32C, a battery 33, and a communication unit 34. The controller 31 is composed of CPUs and the like, and comprehensively controls processing operation of each part of the speed measurement device 30C. Specifically, the controller 31 reads various processing programs stored in a storage section (not illustrated) and performs various kinds of processing in cooperation with the programs.

The speed sensor 32C is a sensor that detects the speed of the target object 2 whose state is to be determined, that is, a parameter related to the speed of movement of the target object 2 as viewed from the speed sensor 32C. As the speed sensor 32C, for example, a sensor using laser, infrared rays, electromagnetic waves, ultrasonic waves, or the like can be used.

The controller 31 acquires a detection result (a parameter related to the speed of the target object 2) of the speed sensor 32C and notifies the determination device 20A of the detection result. Based on the detection result of the speed sensor 32C, the speed of the object 2 as viewed from the odor sensors 12a to 12d is calculated.

The battery 33 supplies power to each unit of the speed measurement device 30C. As the battery 33, a detachable dry cell, a rechargeable battery, or the like is used. The communication unit 34 includes an interface for performing data transmission with the determination device 20A by wireless communication such as Bluetooth (R) and performs data transmission and reception with the determination device 20A in accordance with the communication standard.

Here, the terminal device 40 will be described. The terminal device 40 is a smartphone or the like that can be carried by a user, and includes a controller 41, a display part 42, an operation part 43, a first communication unit 44, a second communication unit 45, a storage section 46, a speaker 47, and a microphone 48.

The controller 41 is composed of a CPU and the like, and comprehensively controls processing operation of each part of the terminal device 40. Specifically, the controller 41 reads various processing programs stored in the storage section 46 and performs various processes in cooperation with the programs.

The display part 42 is configured by a liquid crystal display (LCD) or the like and displays various screens in accordance with an instruction of a display signal input from the controller 41. The operation part 43 includes an operation key and a touch screen stacked on the display part 42, and outputs to the controller 41 an operation signal corresponding to the operation key, and an operation signal corresponding to a position of a touch operation by a user's finger or the like.

The first communication section 44 is wirelessly connected to a communication network including a mobile communication network via a base station or an access point and communicates with an external device connected to the communication network. The second communication unit 45 includes an interface for performing wireless communication such as Bluetooth (R) with an external device such as the determination device 20A. The second communication unit 45 transmits and receives data to and from an external device in accordance with the communication standard.

The storage section 46 is constituted by a nonvolatile semiconductor memory or the like, and stores various processing programs, parameters and files necessary for executing the programs, and the like. The storage section 46 stores an odor detection application program (hereinafter, referred to as an odor detection app) for performing odor detection using the odor detection device 10C.

The speaker 47 converts an electric signal received from an external device via the first communication unit 44 into a sound signal, and outputs the sound. The microphone 48 detects a sound wave, converts the sound wave into an electrical signal, and outputs the electrical signal to the controller 41 and the first communicator 44.

The controller 41 executes the odor detection app, thereby causing the display part 42 to display the determination result of the state of the object 2 received from the determination device 20A via the second communication section 45.

The determination section 24 determines the state of the object 2 from the positional relationship among the odor detection device 10C, the speed measurement device 30C, and the object 2, the speed of the object 2 notified from the speed measurement device 30C, and the value (or the feature amount) outputted from the odor sensor 12 and notified from the odor detection device 10C. In the present embodiment, the determination unit 24 determines whether the object 2 is normal or abnormal. Although details will be described later, the determination section 24 selects an odor determination model 23A corresponding to the speed of the object 2 and determines the state of the object 2 using the selected odor determination model 23A.

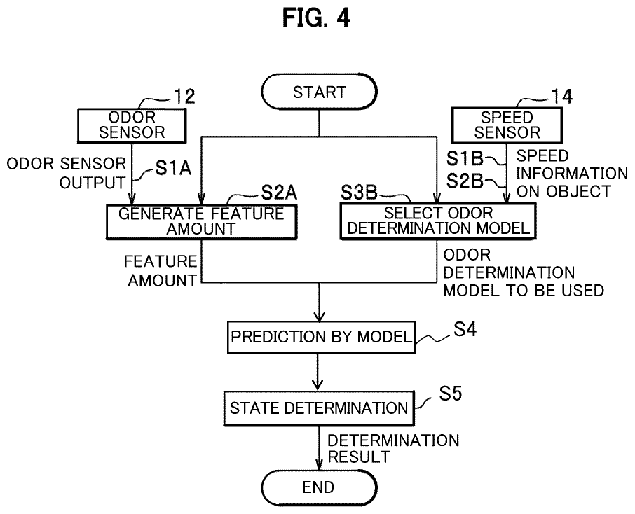

Next, an example of the operation of the odor detection system 1A in FIG. 1 will be described with reference to the flowchart in FIG. 4.

First, the odor sensors 12 (12a to 12d) respond to the odor of the object 2 (step S1A). Next, the determination section 24 acquires values outputted from the odor sensors 12a to 12d and generates a feature amount of the odor based on the acquired values (step S2A). On the other hand, the speed sensor 14 detects the speed of the object 2 (step S1B). Subsequently, the controller 11 calculates, based on the acquired detection result, the speed of the object 2 as viewed from the odor sensors 12a to 12d, and notifies the determination section 24 of the speed (step S2B).

Subsequently, the determination section 24 selects one of the odor determination models 23A (23a˜23c), based on the movement speed information of the object 2 as viewed from the odor sensors 12a˜ 12d, which is calculated by the controller 11 of the odor detection device 10A (step S3B).

Steps S1A and S2A and steps S1B to S3B may be executed at the same time, or one of them may be executed first.

Subsequently, the determination section 24 acquires the feature amount of the odor, predicts the state of the object 2 by using the acquired feature amount and the selected odor determination model 23A (step S4), and determines the state of the object (step S5). That is, the determination unit 24 causes the odor determination model 23A to determine whether the target object 2 is normal or abnormal by inputting the acquired feature amount to the selected odor determination model 23A and obtains a determination result. The determination result is transmitted to the odor detection device 10A via the communication unit 25 and is displayed on the display part 15 by the controller 11.

According to the odor detection system of the first embodiment, since the odor detection system 1A determines the state of the object 2 in consideration of the movement speed of the object 2, it is possible to preferably determine the state of the object 2 using the odor without being affected by the magnitude of the speed of the object 2.

With the odor detection system according to the first embodiment, since the odor determination model 23A determines whether the object 2 is normal or abnormal, the inspection of the object 2 can be suitably performed.

According to the odor detection system of the first embodiment, the odor detection system 1A includes a plurality of odor determination models 23A that have learned, for each of a plurality of speed ranges of the target object 2, values outputted from the odor sensor 12 as explanatory variables and states of the target object 2 as objective variables. Then, the determination unit 24 selects one of the odor determination models 23A based on the calculated speed of the target object 2 and determines the state of the object 2 using the selected odor determination model 23A. Thus, when the speed of the object 2 is low, the odor detection system 1A can reduce the false alarm rate (the rate at which normality is determined as abnormality) by setting the thresholds to be high. Further, when the speed of the target object 2 is high, the odor detection system 1A sets the thresholds to be low so that it is possible to reduce a notification failure rate (a rate at which an abnormality is determined to be normal).

### Second Embodiment

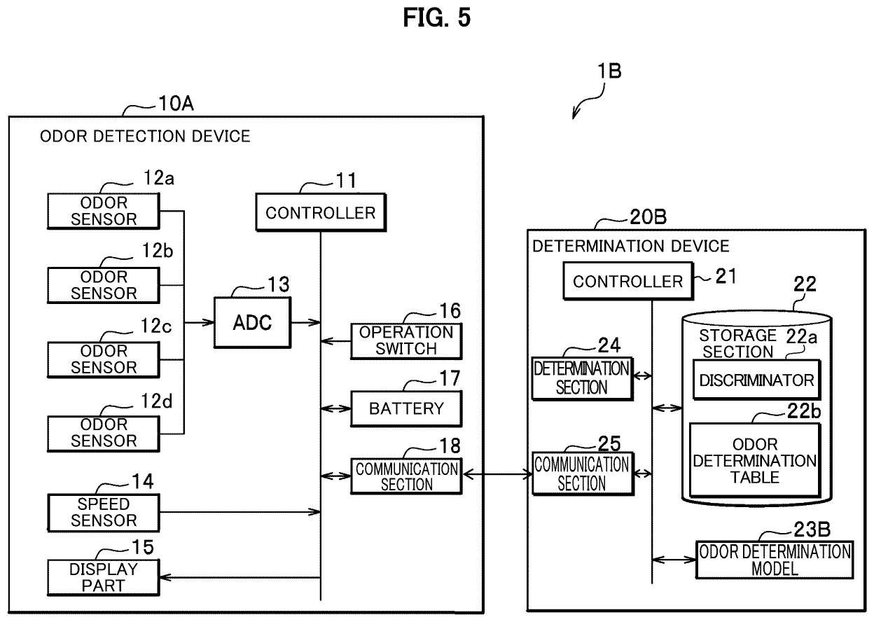

FIG. 5 is a configuration diagram of an odor detection system 1B according to a second embodiment in which a state of an object 2 is determined on the basis of an odor of the object and the speed of the object.

The odor detection system 1B according to the second embodiment is different in that an odor determination model 23B is provided instead of the plurality of odor determination models 23A (23a, 23b, 23c). The odor determination model 23B is described below. The other configuration of the determination device 20B of FIG. 5 is the same as that of the determination device 20A of FIG. 1, and thus the description thereof will be omitted.

The odor determination model 23B is a model that has learned the values outputted by the odor sensors 12a to 12d and the speed of the movement of the object 2 as explanatory variables and the state of the object 2 as an objective variable. The odor determination model 23B calculates an output value related to the state of the object obtained by the machine learning based on the speed of movement of the object 2 and the value (or the feature amount) outputted by the odor sensor 12. Then, the odor determination model 23B determines that the object is normal if the state is less than the threshold value and determine that the object is abnormal if the state is equal to or more than the threshold value.

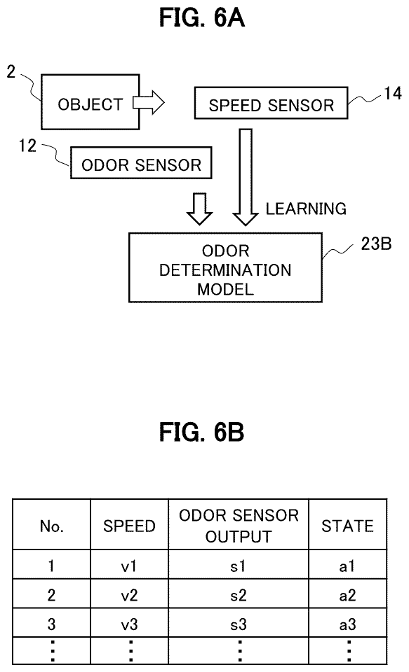

As illustrated in FIG. 6A, the odor sensor 12 performs measurement while changing the speed of the object, and outputs (s1, s2, s3, and . . . ) of the odor sensor 12 at the respective values of speed (v1, v2, v3, and so on) are collected (see FIG. 6B). The odor determination model 23B can handle the distance as a continuous value by inputting the speed as an explanatory variable. At this time, it is assumed that the object 2 is located at a predetermined distance from the odor sensor 12.

In the odor determination model 23B, an optimal threshold is automatically set by inputting the movement speed information, whereby the robustness can be improved. The optimum threshold value is set based on the prediction accuracy of the model, the business value, and the like, and the relationship between the movement speed information and the optimum threshold value can be calculated by regression analysis or the like.

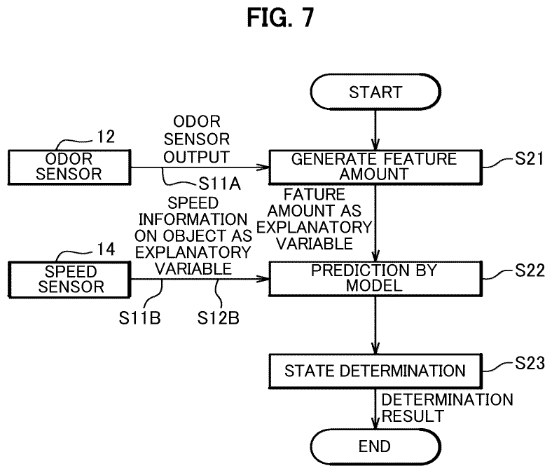

Next, an example of the operation of the odor detection system 1B of FIG. 5 will be described with reference to the flowchart of FIG. 7.

First, the odor sensors 12a to 12d respond to the odor of the object 2 (step S11A). Next, the determination section 24 acquires values outputted from the odor sensors 12a to 12d and generates a feature amount of the odor on the basis of the acquired values (step S21).

On the other hand, the speed sensor 14 detects the speed of the object 2 (step S11B). Subsequently, the controller 11 calculates, based on the acquired detection result, the speed of the object 2 as viewed from the odor sensors 12a to 12d, and notifies the determination section 24 of the speed (step S12B).

Steps S11A and S21 and Steps S11B to S12B may be executed at the same time, or either group of steps may be executed first.

Subsequently, the determination section 24 acquires the detection result of the speed sensor 14, more specifically, the speed of the object 2 calculated by the controller 11, and predicts the state (calculates the state) using the odor determination model 23B in which the acquired speed and the feature amount are used as explanatory variables (step S22).

Subsequently, the determination unit 24 determines the state of the target object 2 based on the generated feature amount of the odor and the determination thresholds by the odor determination model 23B (step S23). That is, the determination unit 24 inputs the acquired feature amount of the odor and the calculated speed of the target object 2 to the odor determination model 23B to predict the state of the target object 2. Then, the determination unit 24 causes the odor determination model 23B to determine whether the object 2 is normal or abnormal and obtains a determination result. The determination result is transmitted to the odor detection device 10A via the communication unit 25 and is displayed on the display part 15 by the controller 11.

According to the odor detection system of the second embodiment, since the optimum thresholds are automatically set by inputting the movement speed information to the odor detection system 1B, the robustness can be improved.

### Third Embodiment

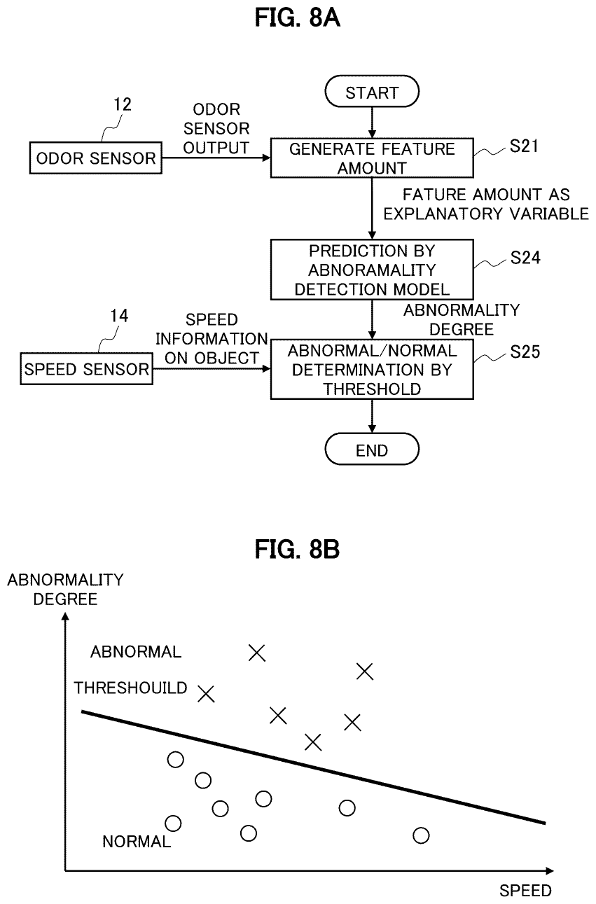

Next, an odor detection system for determining whether the object 2 is normal or abnormal will be described with reference to FIGS. 8A and 8B, wherein the odor detection system employees an abnormality detection model that predicts the degree of abnormality instead of the odor determination model 23B. The system configuration in this case may be the same as that of the odor detection system 1B in FIG. 5.

The abnormality detection model is made by unsupervised learning where the model learns only with normal data and sets optimum thresholds between abnormality data and normal data based on the prediction accuracy and business value of the model. Specifically, the abnormality detection model learns from the feature quantity of the odor sensor and determines whether the state is normal or abnormal based on the output value of the abnormality detection model (an abnormality degree) and the movement speed information. It is also possible to calculate an optimum threshold value for the movement speed information by obtaining from the movement speed information and the abnormality degree at that time the relation with the optimum threshold value by regression analysis or the like as illustrated in FIG. 8B. The boundary may be obtained in the space between the abnormality level and the movement speed by a machine learning model such as an SVM instead of the regression analysis.

An operation example of the odor detection system of the third embodiment will be described with reference to the flowchart of FIG. 8A.

First, the determination section 24 acquires values outputted from the odor sensors 12a to 12d and generates a feature amount of odor based on the acquired values (step S21).

Subsequently, the determination section 24 predicts the abnormality degree by an abnormality detection model in which the feature amount of the odor is used as an explanatory variable.

Next, threshold-value determination of abnormality/normal is performed based on the movement speed information of the object 2 obtained from the detection result of the speed sensor 14 and the abnormality degree that is the prediction result of the abnormality detection model (step S25).

### Fourth Embodiment

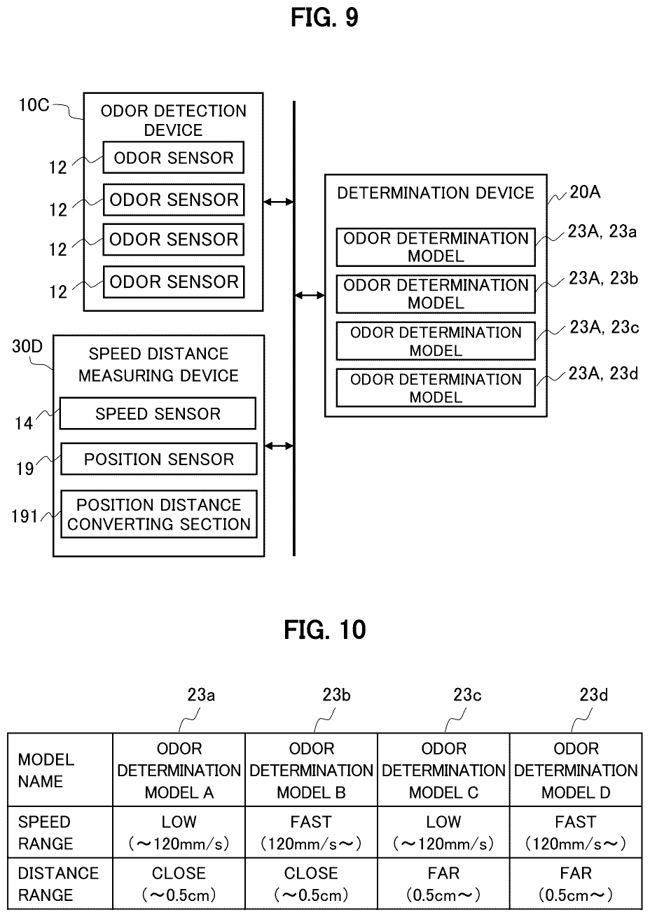

Next, an odor detection system for determining the state of the object 2 will be described with reference to FIGS. 9 to 11, wherein the odor detection system determines the state of the object 2 based on the feature amount of the odor of the object, the position of the object, and the speed of the object. That is, the odor detection system of the fourth embodiment determines the state of the object 2, taking into consideration the information on the positional relationship between the object and the odor sensor calculated from the position of the object.

FIG. 9 is a diagram of a configuration of an odor detection system according to a fourth embodiment.

The odor detection system of the fourth embodiment is configured by adding a position sensor 19 for detecting the position of the object 2 to the odor detection system of the first embodiment illustrated in FIG. 1. Furthermore, the odor detection system according to the fourth embodiment includes a speed sensor 14, a position sensor 19, and a position-to-distance converting section 191, and a speed-distance measuring device 30D that detects the speed and position of the target object 2 and further calculates the distance is configured separately from the odor detection device 10C, similarly to the odor detection system in FIG. 3.

The position sensor 19 for detecting the position of the object 2 may measure the position by a sensor such as a camera or a laser, or the user may directly input the position to the system by hand. Alternatively, the target object 2 may be imaged by a camera or the like, and video information may be analyzed to extract the position and speed of the target object 2. That is, the speed sensor 14 and the position sensor 19 are not limited to specific measuring devices and may be any means capable of calculating the position and the speed of the object 2. Thus, the position distance converting section 191 can calculate the distance between the object 2 and the odor sensor 12 from the position of the object 2. The distance calculated here may be the distance between the odor sensor 12 and the target object 2, or in the case of a production line, the shortest distance when the target object 2 passes near the odor sensor 12.

The determination device 20A, which will be described in more detail later, includes a plurality of odor determination models 23A (23a to 23d). These plurality of odor determination models 23A (23a to 23d) are models that have learned with the output value (or the feature amount) of the odor sensor 12 as an explanatory variable and the state of the object 2 as an objective variable. The odor determination model 23A includes thresholds of the outputs of the odor sensor 12 obtained by machine learning. If the values outputted by the odor sensors 12a to 12c are less than the thresholds, it is determined that the object 2 is normal. If the outputs of the odor sensors 12a to 12c are equal to or greater than the thresholds, the object 2 is determined to be abnormal.

The other configurations of the odor detection device 10C and the determination device 20A are the same as those in FIGS. 1 and 3, and thus the description thereof will be omitted here. The speed-distance measuring device 30D may be configured by adding the position sensor 19 and the position-distance converting section 191 to the speed-measuring device 30C in FIG. 3, and the other configuration of the speed-distance measuring device 30D may be similar to that of the speed-measuring device 30C.

FIG. 10 illustrates odor determination models corresponding to combinations of the speed range and the distance range of the target object 2 and describes a method of generating the odor determination models.

Specifically, the odor determination model A (odor determination model 23a) is a model obtained by machine learning based on the outputs (or features) of the odor sensor 12 with respect to the target object 2 that moves in a slow speed range (˜120 mm/s) and at a position in a close distance range (˜0.5 cm) as viewed from the odor sensor 12.

Similarly, the odor determination model B (odor determination model 23b), the odor determination model C (odor determination model 23c), and the odor determination model D (odor determination model 23d) are models obtained by machine learning based on the outputs (or feature amounts) of the odor sensor 12 with respect to the object 2 at the speed in the speed range and the positions in the range of distances illustrated in FIG. 10.

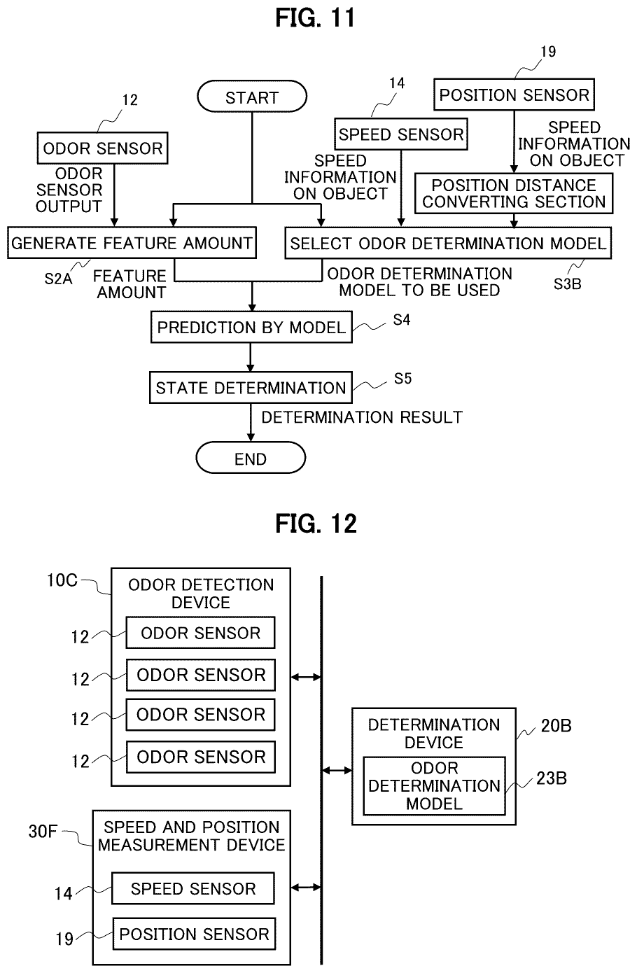

Next, an example of the operation of the odor detection system according to the fourth embodiment in FIG. 9 will be described with reference to the flowchart in FIG. 11.

First, the determination section 24 acquires values outputted from the odor sensors 12 (12a to 12d) and generates a feature amount of odor based on the acquired values (step S2A).

On the other hand, a controller 31 (not illustrated) of the speed and distance measurement device 30D detects the speed of the object by the speed sensor 14 and calculates the speed of the object 2 as viewed from the odor sensor 12 and notifies it to the determination section 24. Further, the controller 31 detects the position of the target object by the position sensor 19 and calculates the distance of the object 2 from the odor sensor 12 with the position-to-distance converter 191 and notifies it to the determination section 24. The position sensor 19 and the position-to-distance converter 191 function as a distance calculation section that calculates the distance between the object 2 and the odor sensor 12.

Subsequently, the determination section 24 selects one of the odor determination models 23A (23a to 23d) on the basis of the movement speed information and the distance information on the object 2 as viewed from the odor sensor 12, wherein the movement speed information and the distance information are notified from the speed-distance measuring device 30D (step S3B).

Subsequently, the determination unit 24 acquires the feature amount of the odor, predicts the state of the target object 2 using the acquired feature amount and the selected odor determination model 23A (step S4), and performs the state determination of the predicted state (step S5). That is, the determination unit 24 causes the odor determination model 23A to determine whether the target object 2 is normal or abnormal by inputting the acquired feature amount to the selected odor determination model 23A and obtains a determination result.

### Fifth Embodiment

Next, with reference to FIGS. 12 to 14, a description will be given of an odor detection system that is different from the fourth embodiment and that determines the state of the object 2 on the basis of the feature amount of the odor of the object, the position of the object, and the speed at which the object moves.

FIG. 12 is a configuration diagram of an odor detection system according to a fifth embodiment.

The odor detection system according to the fifth embodiment is different from the odor detection system according to the fourth embodiment in that the determination device 20B includes a single odor determination model 23B instead of the plurality of odor determination models 23A (23a, 23b, 23b, and 23d), similarly to the odor detection system according to the second embodiment illustrated in FIG. 5.

The odor determination model 23B according to the fifth embodiment is a model that has learned the outputs (or feature amounts) from the odor sensors 12a to 12d, the speed of movement of the object 2, and the position of the object 2 as explanatory variables, and the state of the object 2 as an objective variable. The odor determination model 23B calculates an outputted value relating to a state of the object 2 obtained by machine learning based on the speed of movement of the target object 2, the position of the target object 2, and the outputted value (or the feature amount) of the odor sensor 12. Then, the odor determination model 23B determines that the object is normal if the state is less than the threshold value and determines that the object is abnormal if the state is the threshold value or more.

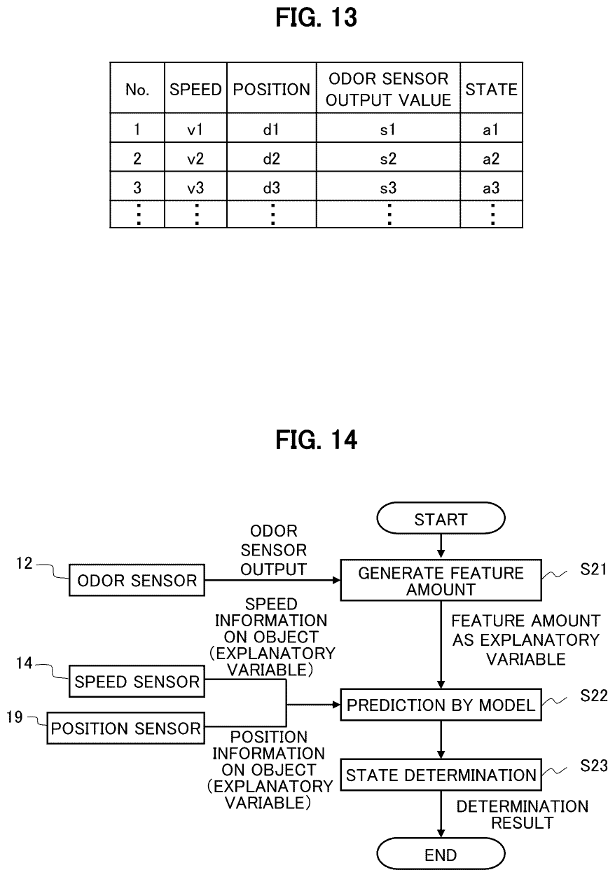

As illustrated in FIG. 13, the odor determination model 23B changes the speed (v1, v2, v3, and . . . ) and the position (d1, d2, d3, and . . . ) of the object 2, and collects, for each combination of the speed and the position, the values (s1, s2, s3 and . . . ) outputted from the odor sensor 12 and the states of the object 2 (a1, a2, a3).

Next, an operation example of the odor detection system of the fifth embodiment of FIG. 12 will be described with reference to the flowchart of FIG. 14.

First, the determination section 24 acquires values outputted from the odor sensors 12a to 12d and generates a feature amount of odor based on the acquired values (step S21).

On the other hand, the controller 31 (not illustrated) of the speed and position measuring device 30F detects the speed of the object 2 by the speed sensor 14. Then, the controller 31 calculates the speed of the object 2 as viewed from the odor sensor 12 and notifies the determination unit 24 of the speed of the object 2. Furthermore, the controller 31 detects the position of the object by the position sensor 19, calculates the position of the object 2 as viewed from the odor sensor 12, and notifies the determination section 24 of the calculated position.

Subsequently, the determination section 24 acquires the speed and the position of the target object 2 as viewed from the odor sensor 12, which are notified from the speed and position measuring device 30F. Next, the determination section 24 predicts the state (calculates the state) using the odor determination model 23B in which the acquired speed and position of the object 2 and the feature amount of the odor are defined as explanatory variables (step S22).

Subsequently, the determination section 24 determines the state of the target object 2 based on the generated feature amount of the odor and the determination thresholds by the odor determination model 23B (step S23). That is, the determination section 24 predicts the state of the target object 2 from the acquired feature amount of the odor and the calculated speed and position of the target object 2 by the odor determination model 23B, determines whether the target object 2 is normal or abnormal from the predicted state, and obtains the determination result.

According to the odor detection system of the fourth embodiment or the fifth embodiment, since the odor determination models are learned with more explanatory variables and the state is predicted with the odor determination models, the state of the object 2 can be determined with high accuracy.

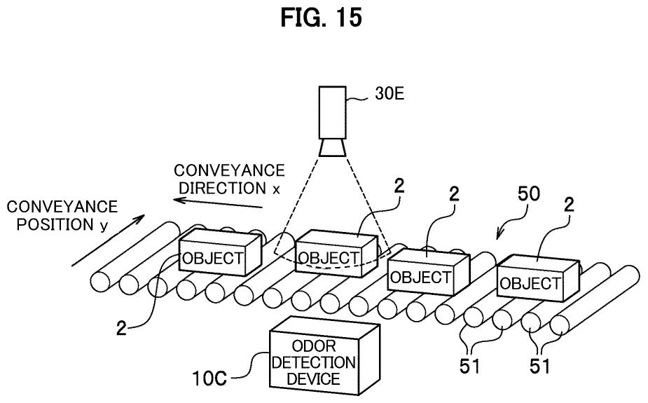

Next, specific application examples of the odor detection system of the fourth embodiment or the odor detection system of the fifth embodiment will be described. FIG. 15 is an external view of a production line to which the odor detection system of the embodiments is applied, where the object 2 is conveyed by a conveyance device 50. The odor detection system determines whether or not the object 2 being conveyed is damaged by the odor.

The conveying device 50 includes a plurality of rollers 51 and conveys the object 2 as a product placed on the rollers 51 in the conveyance direction x by rotating the rollers 51.

The odor detection device 10C is installed below the conveyance device 50. Furthermore, although not illustrated in FIG. 15, the speed and position measurement device 30F of FIG. 9 or FIG. 12 is arranged at the same coordinates as the odor detection device 10C in the conveyance direction x and at a position that is on a side of the conveyance device 50 and at the same height as the object 2.

The camera 30E is disposed above the conveyance device 50, and in the present embodiment, right above the odor detection device 10C. The camera 30E only needs to be able to image the object 2 and detect the position and the movement speed of the object 2 and may be a sensor such as a LiDAR sensor, a laser sensor, or an ultrasonic sensor.

The camera 32E functions as a sensor for detecting the position and the speed of the target object 2 whose state is to be determined, that is, the camera 32E is the position sensor 19 and the speed sensor 14. Specifically, the speed and position measurement device 30F extracts the object 2 from the captured image of the camera 32E and obtains the position of the object 2 viewed from the camera 32E. Then, from the positional relationship between the camera 32E and the odor detection device 10C and the position of the target object 2 viewed from the camera 32E, the position of the object 2 viewed from the odor detection device 10C is obtained and used as the position information of the object 2.

Furthermore, the speed and distance measurement device 30F obtains the movement speed of the object 2 as viewed from the camera 32E from a change in the position of the extracted object 2 between frames of the captured images. Then, the movement speed of the object 2 as viewed from the odor detection device 32E is obtained from the positional relationship between the camera 10C and the odor detection device 32E and the movement speed of the object 2 as viewed from the camera 10C, and is set as the movement speed information of the object 2.

Thus, even in a production line in which the placement position of a product as the object 2 changes and the conveyance speed of the conveyance apparatus 50 changes, breakage of the product can be detected with high accuracy.

Furthermore, the present invention is not limited to the above-described examples and includes various modification examples. The above-described examples have been described in detail for easy understanding of the present invention, and the present invention is not necessarily limited to those having all the configurations described above. Furthermore, part of the configuration of an embodiment can be replaced with the configuration of another embodiment, and the configuration of another embodiment can be added to the configuration of an embodiment.

### DESCRIPTION OF REFERENCE NUMERALS

1A, 1B, 1C ODOR DETECTION SYSTEM
2 OBJECT
11 CONTROLLER (SPEED CALCULATION SECTION)
12, 12A, 12B, 12C, 12D ODOR SENSOR
14 SPEED SENSOR
23A, 23B, 23C ODOR DETERMINATION MODEL
23B ODOR DETERMINATION MODEL
24 DETERMINATION SECTION
30C SPEED MEASUREMENT DEVICE
30D SPEED AND DISTANCE MEASURING DEVICE
30F SPEED •POSITION MEASURING DEVICE
31 CONTROLLER (SPEED CALCULATION SECTION)
32C SPEED SENSOR

What is claimed is:

1. An odor detection system, comprising:
an odor sensor that reacts to an odor of an object;
a speed calculation section that calculates movement speed of the object as viewed from the odor sensor; and
a determination section that determines a state of the object based on an output value of the odor sensor and the movement speed of the object.

2. The odor detection system according to claim 1, wherein the determination section determines whether or not the object is abnormal.

3. The odor detection system according to claim 2, wherein the determination section determines whether or not the object is damaged.

4. The odor detection system according to claim 1, further comprising:
a plurality of odor determination models that have learned the output value of the odor sensor as an explanatory variable and the state of the target object as an objective variable for each of a plurality of values of movement speed, wherein
the determination section selects one of the odor determination models based on the movement speed calculated by the speed calculation section and determines the state of the object using the selected odor determination model.

5. The odor detection system according to claim 1, further comprising:
an odor determination model that has learned the movement speed and the output value of the odor sensor as explanatory variables and the state of the object as an objective variable; wherein
the determination section determines the state of the object based on the movement speed calculated by the speed calculation section and the output value of the odor sensor, using the odor determination model.

6. The odor detection system according to claim 1, further comprising:
a distance calculation section that calculates a distance between the object and the odor sensor, wherein
the determination section determines the state of the object based on the output value of the odor sensor, the movement speed of the object as viewed from the odor sensor, and the distance between the object and the odor sensor.

7. The odor detection system according to claim 6, further comprising:
a plurality of odor determination models that have learned an output value of the odor sensor as an explanatory variable and a state of the target object as an objective variable for each of a plurality of sets of the movement speed and the distance; wherein
the determination unit selects one of the odor determination models based on the movement speed calculated by the speed calculation section and the distance and determines the state of the object using the selected odor determination model.

8. The odor detection system according to claim 6, further comprising:
an odor determination model that has learned the movement speed, the distance, and the output value of the odor sensor as explanatory variables and the state of the object as an objective variable; wherein
the determination section determines the state of the object based on the movement speed calculated by the speed calculation section, the distance calculated by the distance calculation section, and the output value of the odor sensor by using the odor determination model.

9. The odor detection system according to claim 1, further comprising
a plurality of the odor sensors having mutually different characteristics to react to odors, wherein
the determination section identifies the object on the basis of output values from the plurality of the odor sensors.

10. An odor detection method, performed by a system including an odor sensor, a speed sensor, a speed calculation section, and a determination section, the method allowing the system to perform:
causing the odor sensor to react to an odor of an object;
causing the speed sensor to detect movement speed of the object;
causing the speed calculation section to calculate movement speed of the object as viewed from the odor sensor, and
causing the determination section to determine a state of the object based on an output value of the odor sensor and the movement speed of the target object as viewed from the odor sensor.

11. A non-transitory computer-readable storage medium storing a program causing a computer to perform:
acquiring an output value of an odor sensor that has reacted to an odor of an object;
acquiring movement speed of the object detected by a speed sensor;
calculating movement speed of the object as viewed from the odor sensor with a speed calculation section; and
determining with a determination section a state of the object based on an output value of the odor sensor and the movement speed of the target object as viewed from the odor sensor.
